(19) United States
(12) Patent Application Publication
(10) Pub. No.: US 2015/0011830 A1
(43) Pub. Date: Jan. 8, 2015

(54) TIP ACTUATED DISPOSABLE ENDOSCOPE

(71) Applicants: Ian W. Hunter, Lincoln, MA (US); Yi Chen, St. Charles, MO (US); Jillian M. Oliveira, West Chester, PA (US)

(72) Inventors: Ian W. Hunter, Lincoln, MA (US); Yi Chen, St. Charles, MO (US); Jillian M. Oliveira, West Chester, PA (US)

(73) Assignee: MASSACHUSETTS INSTITUTE OF TECHNOLOGY, Cambridge, MA (US)

(21) Appl. No.: 14/355,490

(22) PCT Filed: Aug. 25, 2011
(86) PCT No.: PCT/US2011/049167

§ 371 Date: Apr. 30, 2014

Related U.S. Application Data
(60) Provisional application No. 61/480,735, filed on Apr. 29, 2011.
(60) Provisional application No. 61/377,765, filed on Aug. 27, 2010.

(51) Int. Cl. A61B 1/00; A61B 1/005

(52) U.S. Cl. CPC A61B 1/0016; A61B 1/0052; A61B 1/0055
USPC 600/118

(57) ABSTRACT

The present invention relates to an endoscope having a motorized assembly to control the distal end of an endoscope. Preferred embodiments can include disposable and reusable components. The system reduces the force need to control the tip of the endoscope and thereby reduce the risk of perforation′.

### CROSS-REFERENCE TO RELATED APPLICATION

This application claims the benefit of U.S. Provisional Application No. 61/377,765, filed Aug. 27, 2010 and U.S. Provisional Application No. 61/480,735 filed on Apr. 29, 2011, the entire contents of these applications being incorporated herein by reference.

### BACKGROUND OF THE INVENTION

An endoscope is an instrument used to examine the interior of a patient's body. Endoscopes are used for diagnosis and/or treatment in a number of areas, including, for example, in the gastrointestinal or respiratory systems. Most flexible endoscopes utilize cable systems to manipulate the position of the tip to provide directional control during placement of the endoscope within a human or animal body. The user typically manually adjusts the position of the cables from the proximal end of the endoscope, which consequently subjects the cable system to capstan friction forces throughout the body of the endoscope. This defines the amount of force that must be applied to the cable system and tends to increase the amount of force applied to the tip of the endoscope. Higher lateral tip forces increase the risk of perforation of the body lumen in which the endoscope is being moved. It is also advantageous for the endoscope to have longitudinal stiffness during insertion, yet be flexible during retraction to reduce the risk of endoscope looping and perforation.

Another important factor in endoscope design is to minimize the risks and costs associated with the sterilization of components. There is, consequently, a continuing need for improvements in endoscope design for both medical and industrial use.

### SUMMARY OF THE INVENTION

The present invention relates to a system for directional control of an endoscope. A preferred embodiment of the invention comprises an endoscope system having a steerable distal end to facilitate insertion into body lumens. The endoscope system can employ a motorized control system to actuate deflection of the distal end of the endoscope. By locating the actuator used to control the tip at or near the distal end of the endoscope, tip forces and input force variation can be reduced. When the cable system is actuated from the proximal end of the endoscope, as in conventional steerable systems, the cable system is subject to capstan friction forces throughout the endoscope body. This increases the maximum force that the operator must use at the proximal end, thereby increasing the range of forces that can be seen at the tip of the endoscope. However, with the distal actuator placed at or near the tip, the capstan friction is lower so that the maximum force at the tip can be more tightly controlled. The removal of the cables needed for tip control from the proximal body of the endoscope also reduces the complexity and cost of a disposable portion of the system. Further, the system is capable of turning speeds in excess of 400 degrees per second for fast response and control. In a further preferred embodiment, the system can use higher torque in order to bend flexible biopsy tools inserted through an endoscope channel or lumen.

A preferred embodiment of the invention includes a disposable elongate body section that is detachable from a handle with a connector. The elongate body section is adapted for at least partial insertion in a human or animal body. The elongate body is attached at the distal end to a motorized section that includes a motor to actuate deflection of a flexible section. The distal end of the flexible section can be attached to a distal housing that can contain devices or instruments for particular procedures. A preferred embodiment of the distal housing can include a light source, angular displacement sensors such as gyroscopes, and an electronic image sensor or camera for visualization. The distal section can be detached and cleaned for reuse or can be readily replaced if damaged.

The system is designed to limit the force applied by the tip to the tissue when it is deflected. The torque that is applied by the motor on the tip has an upper limit that can be selected integral with the design, or it can be adjusted as a programmable parameter by the user. Alternatively, a further preferred embodiment measures the force imparted by the tip on the tissue, which can be displayed to the user. Additionally, a haptic feedback system can used to give the user a tactile sense of the level of force being imparted.

### BRIEF DESCRIPTION OF THE DRAWINGS

FIG. 1 schematically illustrates a preferred embodiment of the invention.

FIGS. 2A and 2B illustrate preferred embodiments of the motor assembly for tip control in accordance with preferred embodiments of the invention.

FIG. 3A illustrates an endoscope system in accordance with the invention.

FIG. 3B illustrates an electronic control system of a preferred embodiment of the invention.

FIG. 3C illustrates a further preferred embodiment of an electronic control system in accordance with the invention.

FIG. 3D illustrates a force sensor and haptic control system used in accordance with the invention.

FIG. 4 illustrates and end view of a turning module.

FIGS. 5A and 5B illustrate perspective and end views of an endoscope body module.

FIG. 6 is a cross-sectional side view of a motor module.

FIGS. 7A and 7B show sectional views of the gear box assembly.

FIGS. 7C and 7D show cutaway perspective views of gear assemblies used in preferred embodiments of the invention.

FIGS. 8A-8C illustrate perspective and sectional views of a motor converter, respectively.

FIGS. 9A-9C illustrate perspective and sectional views of a tip to turning converter, respectively.

FIGS. 10A-10C illustrate perspective and sectional views of the body to handle converter, respectively.

FIGS. 11A and 11B show side and end views of tip connections.

FIGS. 12A and 12B show side and end views of the connectors at the motor assembly.

FIGS. 13A and 13B show perspective and end views of the tip assembly.

FIG. 14 illustrates forces acting on the tip assembly of an endoscope.

FIGS. 15A and 15B illustrate static normal forces as a function of bending angle and bending angle as a function of time for different motor torque levels, respectively.

FIG. 16A illustrates an alternative embodiment of a motor module section for an endoscope system.

FIG. 16B illustrates a worm gear and a wheel included in the motor module section of FIG. 16a.

FIG. 17A is a diagram illustrating an exemplary bending angle of an endoscope, with and without a biopsy tool inserted, as a function of pulling force.

FIG. 17B is a diagram illustrating an exemplary stall force as a function of voltage for the endoscope of FIG. 17a.

FIG. 17C is a diagram of an exemplary stall speed as a function of pulling force for the endoscope of FIG. 17a.

FIG. 18 illustrates an alternative embodiment of an architecture for an endoscopic robotic platform.

FIG. 19 illustrates a sensor and actuator architecture for the endoscopic robotic platform of FIG. 18.

FIG. 20 illustrates the nominal alignment of sensors relative to a motor module control coordinate system for the sensor and actuator architecture of FIG. 19.

FIG. 21A is a diagram illustrating measured and simulated bending angles relative to the motor module control coordinate system of FIG. 20.

FIG. 21B is a diagram illustrating measured and simulated values for a voltage command from a controller in the sensor and actuator architecture of FIG. 19

### DETAILED DESCRIPTION OF THE INVENTION

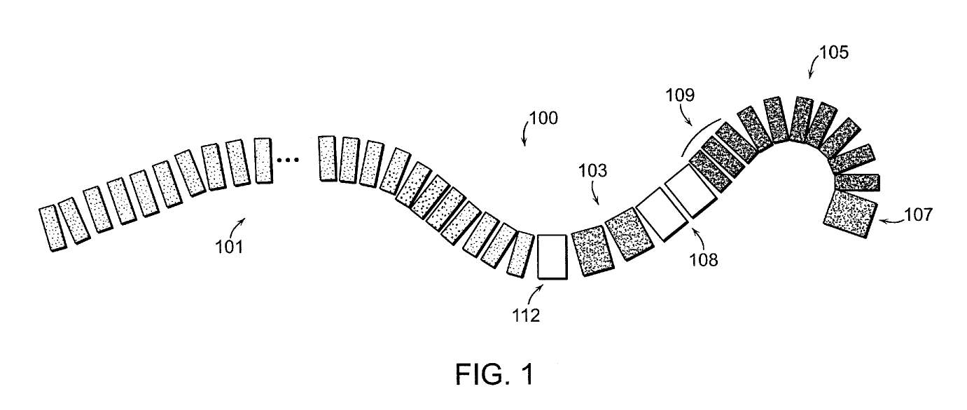

FIG. 1 is a schematic illustration of an endoscope 100 according to one embodiment of the invention. In one embodiment, the endoscope 100 has a modular design including a plurality of sections or modules, including body section 101, motor module 103, turning section 105 and distal tip module 107. Various other components can be included, such as, for example, one or more converters 109 for converting between various sections, and a safety module 108 that can cut the control cables for the turning section 105 in case of a malfunction. One or more connectors 112 can be employed to connect the various sections and/or modules of the endoscope 100.

The modular design is preferably selected to reduce manufacturing cost and permit simple adjustment in length. In one embodiment, the modular design allows for the disposal of the most difficult-to-clean sections of the endoscope, which typically includes the body section 101, motor module 103 and turning section 105. In one preferred embodiment, all components except the tip module 107 can be designed to be low cost and disposable. The tip module 107 is generally non-disposable and can be separately sealed, thereby mitigating contamination.

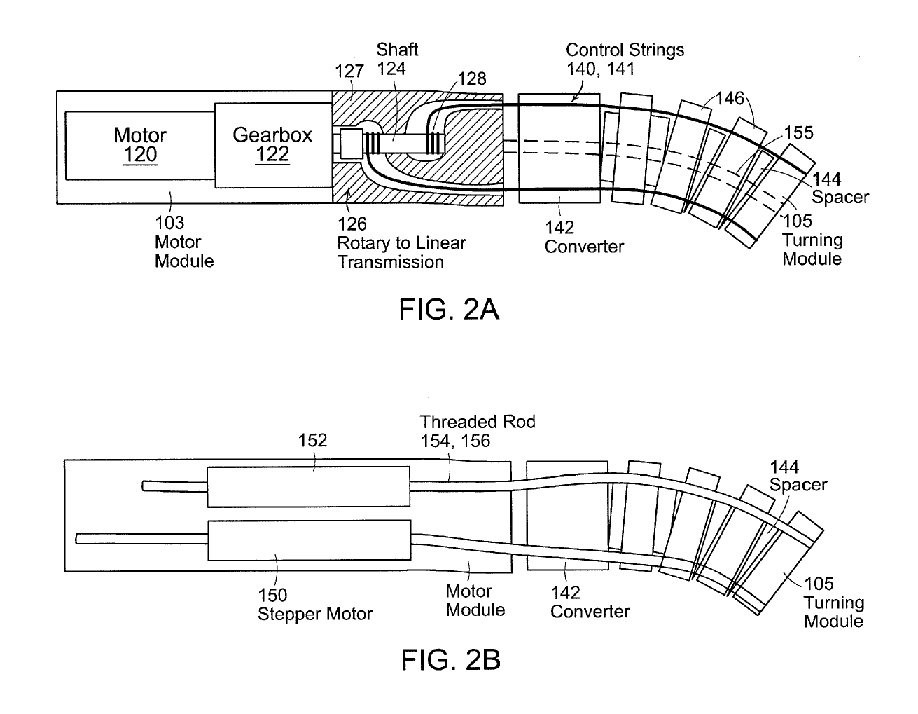

FIG. 2A illustrates a preferred embodiment of a motor module in cross-section. As illustrated in FIG. 2A, the motor module can comprises a motor 120 coupled to a gear assembly 122 that rotates a shaft 124 that is connected at distal end 128 to a first control wire 140 and at a proximal end 127 to a second control wire 141 (or the opposite ends of a single wire). By using a rotary to linear transmission 126, a single motor can control movement in opposite directions. A converter 142 connects the motor module to a plurality of turning module elements 146 connected by spacers 144. As described in detail below, a force sensor 155, such as a strain gage, can be incorporated into the flexible section 105. Position sensors and/or gyroscopes can also be used.

As shown in FIG. 2B, the motor module can include stepper motors 150, 152 coupled to flexible threaded rods 154, 156 that operate the turning module. Additional details for remote actuation can be found in Peirs et al., “A Miniature Manipulator for Integration in a Self-Propelling Endoscope,” Sensors and Actuation A, Vol. 92, PP. 343-349, 2001, the entire contents of which is incorporated herein by reference.

The endoscope 100 can include at least one inner lumen that extends continuously along the length of the endoscope 100 and through the tip module 107 to allow use of a guidewire, tool or other device. A plurality of smaller channels extend from the motor module 103 through the turning section 105 and house the control cables that control the bending action of the endoscope 100, as described in further detail below. The motor module 103 houses the motor 120 that drive the bending motion of the endoscope 100.

In certain embodiments, the body section 101 of the endoscope 100 includes a variable stiffness feature. Variable stiffness allows the endoscope 100 to be stiffened when it enters the patient's body so that it can be easily inserted and advanced. When the endoscope exits the body, the stiffness can be reduced so that it can be easily withdrawn from the body without causing damage and so that loops in the endoscope can be resolved. In addition, the variable stiffness can be adjusted below the perforation threshold for patients that may be more susceptible.

The safety module 108 can be utilized to cut the control cables in the event that the motor(s) malfunction while the tip is bent. This allows the endoscope to be withdrawn without causing injury. Since the turning section 105 is included in the disposable portion of the endoscope, cutting the control cables is not problematic. If higher holding forces are necessary for the tip of the endoscope, additional module(s) can be added to help hold the cables in place.

The distal section or module 107 can include an imaging detector, such as a CCD or CMOS imager and a light source(s) such as one or more light emitting diodes (LEDs) or lasers. Additional instruments for biopsy and other procedures can be passed down the inner lumen.

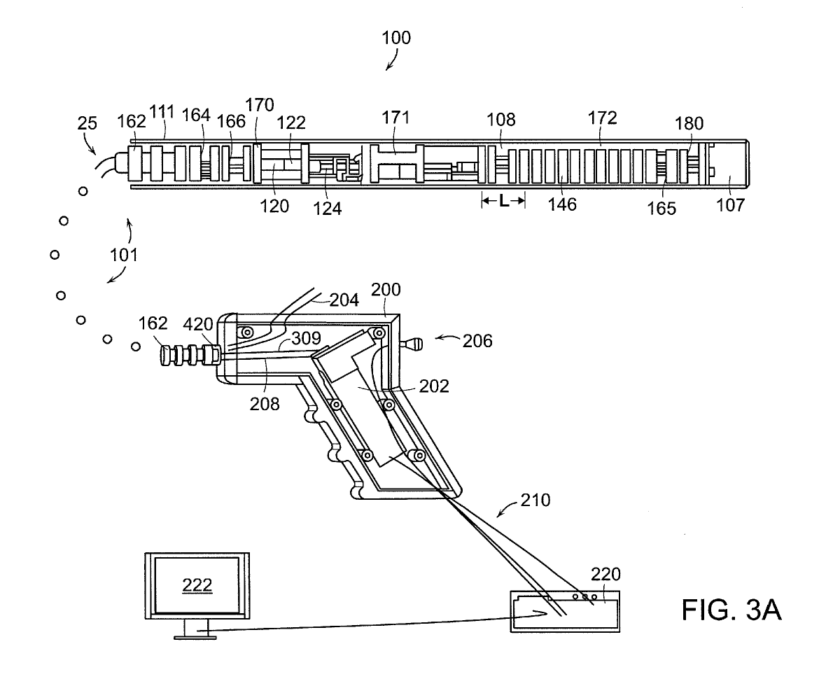

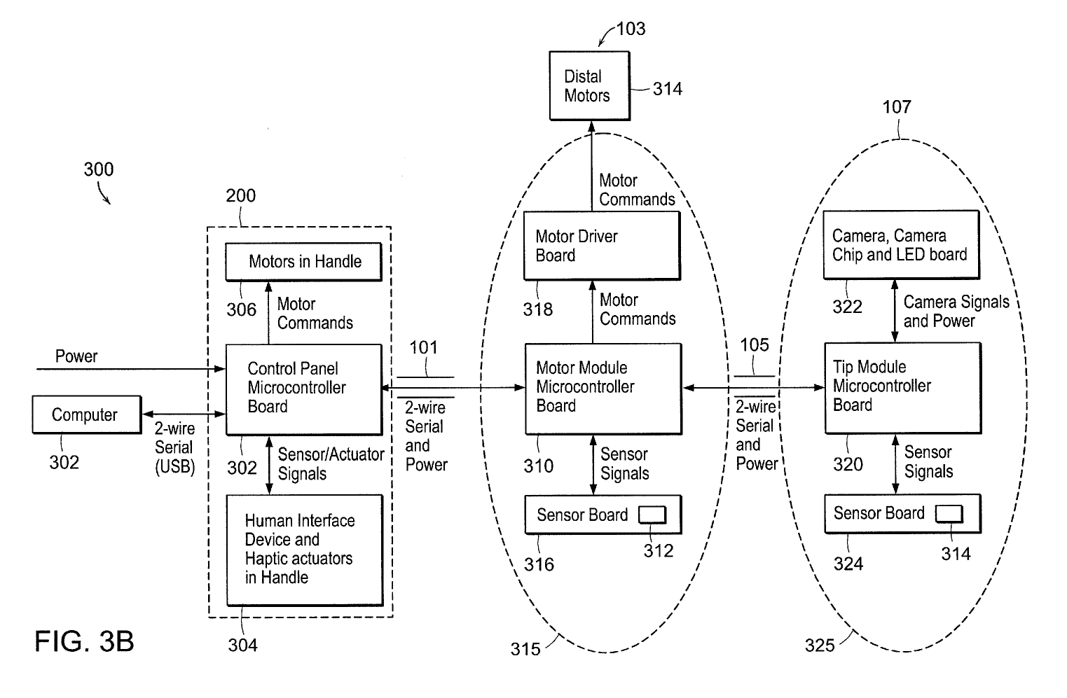

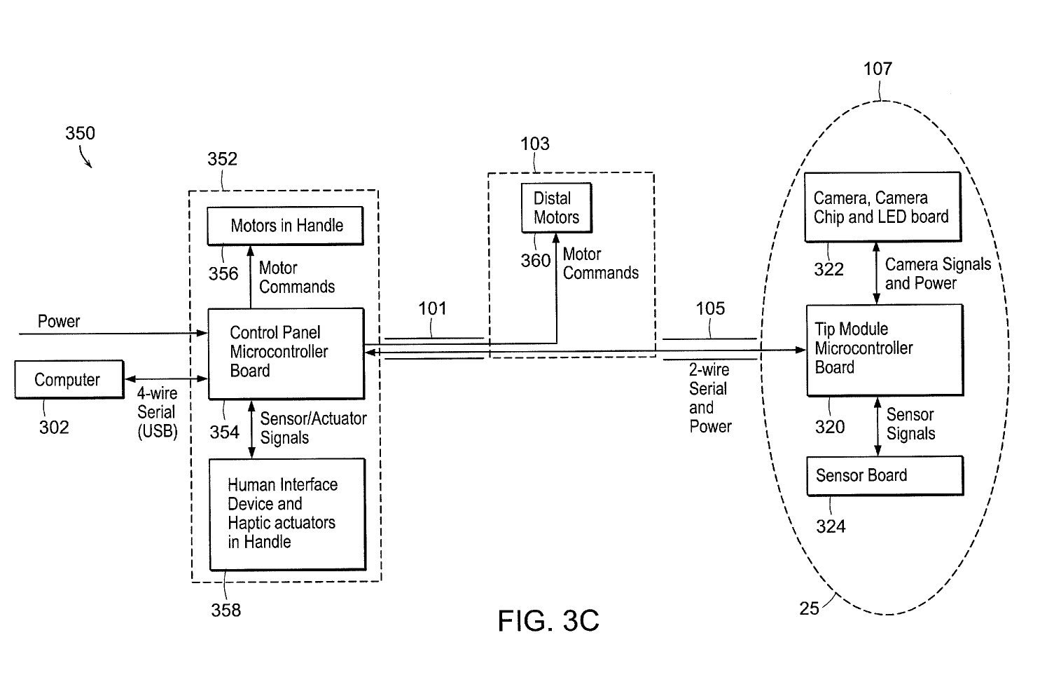

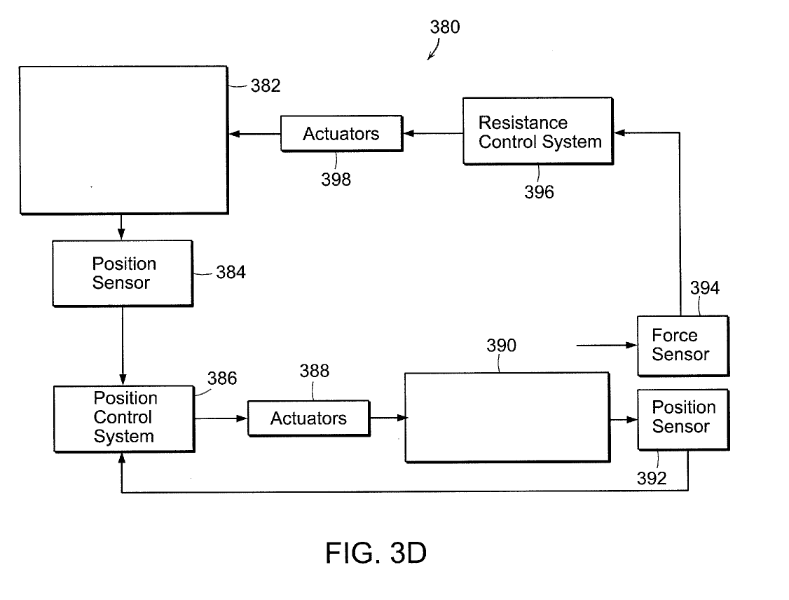

FIG. 3A is a cross-sectional view of an endoscope 100 according to one embodiment of the present invention. As in the embodiment of FIGS. 1 and 2, the endoscope 100 includes a body section 101, a motor module 103 in this embodiment having a first motor 120 and a second motor 171, a turning section 105, and a tip module 107. In a preferred embodiment, these components are housed within a flexible outer endoscope sheath 172, which can be comprised of a suitable bio-compatible material. The proximal end of the body section 101 is attached to a handle 200 that allows the operator to easily manipulate the endoscope 100. The disposable body section can also have a separate outer sheath 111. The inner lumen 25 of the endoscope 100 enters the endoscope through the handle 200 in this embodiment. Other channels, such as an air/water channel, as well as wires for providing signals and power to the motor module 103 and tip module 107, for example, can also enter the endoscope 100 through the handle 200.

It is particularly advantageous to reduce the cable length L extending from the distal end of the motor section 103 to the proximal end of the flexible section 105. The distance L is preferably less than 20 mm, and more preferably is less than 10 mm. For example, length L can be in a range of 1 to 10 mm. Larger lengths can be used to accommodate a safety module, for example. If two motors are positioned longitudinally along the length of the motorized section, the cable system for the proximal motor can be longer than the cable system for the distal motor. In the case of a motorized section with 4 motors, such as two pairs of stepper motors, there can be two pairs of cables having different lengths.

The endoscope 100 also includes at least one connector that allows various segments of the endoscope to be attached to and detached from one another. In one embodiment, connector 165 connects the tip module 107 to an endoscope sub-assembly that includes the body section 101, motor module 103 and turning section 105. In one preferred embodiment, the tip module 107 is reusable, and is removed from the sub-assembly by detaching the tip 107 at the connector 165. The sub-assembly comprising the turning section 105, motor module 103 and body section 101 is disposable, and can be discarded after use.

In another embodiment, connector 164 attaches the body section 101 to a sub-assembly that includes at least the motor module 103 and turning section 105. In this embodiment, the body section 101 can be separated from the rest of the endoscope and either sterilized for reuse, or discarded, as appropriate. Similarly, the motor module 103 and turning section 105 can be reusable, though generally these components will be disposable and discarded after use.

Although the embodiment of FIG. 3A shows two connectors 164, 165 it will be understood that any number of connectors can be used in alternative embodiments of the invention. In general, the connectors are used to separate at least one section and/or module of the endoscope that is reusable from at least one section and/or module that is disposable.

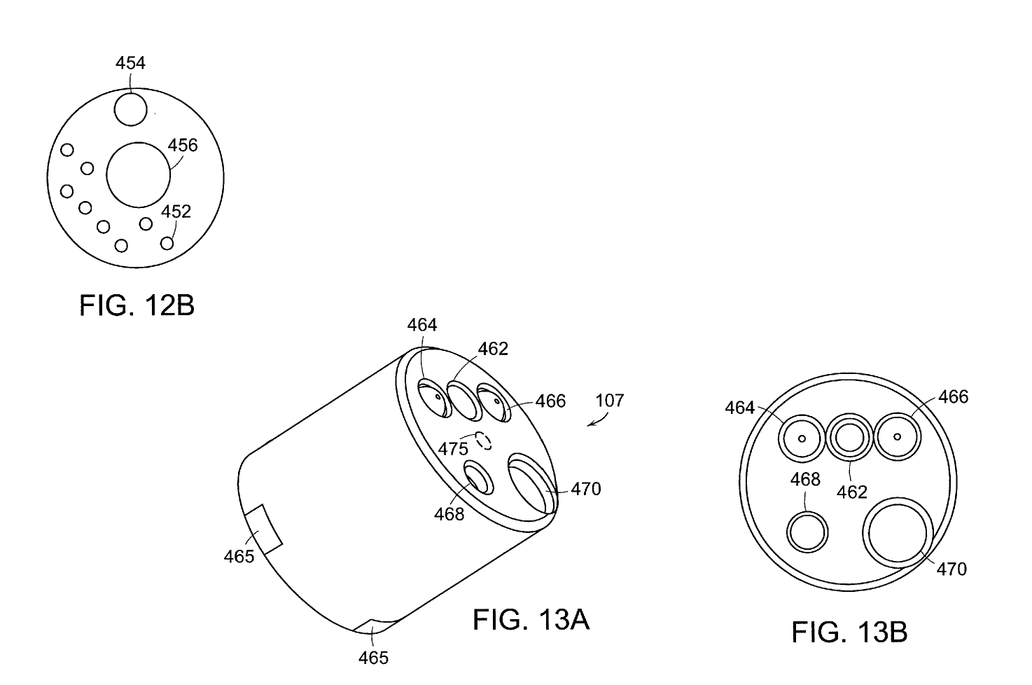

The connectors 164, 165 generally utilize a plug and socket that provides both physical and electrical connection between adjoining sections and/or modules of the endoscope 100. The connectors also include openings to allow the passage of channels, such as the inner lumen, between the adjoining sections and/or modules of the endoscope 100. Examples of connectors 164, 165 according to one embodiment are shown in FIGS. 11A, 11B and 12A, 12B, respectively.

In the embodiment of FIG. 3A, a converter can be provided at the interface of the handle 200 and body section 101 to facilitate routing of wires and channels from the handle 200 into the body section 101. Similar converters 166, 168, 180 are used to route the various wires and/or channels from the body section 101 to the motor module 103, from the motor module 103 to the turning section′ 105, and from the turning section 105 to the tip module 107.

In the embodiment of FIG. 3A, the handle 200 is connected to a control panel 220 by one or more wires 210. The control panel 220 can be used to control the settings and operation of the endoscope 100. In addition, an interface actuator device 206, such as a joystick, is provided on the handle 200, to allow the operator to control specified functions of the endoscope 100. In one embodiment, the interface device 206 is used to actuate the motor module 103 and turning section 105 to control the steering of the endoscope tip 107. In other embodiments, the interface device 206 is provided on the user interface 220, or is a separate, stand-alone device.

A computer with the user interface, includes memory and a processor for executing stored instructions, and controls the overall operation of the endoscope 100. The computer can generate control signals that control the operation of one or more motors within motor module 103 that in turn drive control cables within the turning section 105 and steer the tip module 107 in the desired direction. The motor control signals can be generated in response to user commands from an interface device 206 or control panel 220, and in some embodiments, can be generated in response to feedback signals received from sensing devices on the endoscope. The computer can also control the imaging functions for display 222 and motion of the endoscope 101, and receives and processes image signals received from an image sensing device, such as a CCD camera, located in the tip module 107. The computer can further include a display device for displaying images received from the endoscope tip 107.

In a preferred embodiment, the control system 300 can be configured as shown in FIG. 3B. The computer 302 that is connected to the handle 200, which houses a controller circuit board 302 that sends motor commands to motors within the handle, sends and receives signals to the interface and haptic actuators 304 in the handle 200. Power and control cables through the disposable body section 101 of the endoscope are connected to the motorized section 103, which in this embodiment, is reusable and sealed within motor section body 315. A motor module controller board 310 can drive a separate motor driver board 318, which drives the distal motors 319 (which can be within or outside body 315). The controller board can receive signals from the sensor board 316, which can also incorporate gyroscope 312 that measures relative angle difference in combination with gyroscope 314 mounted on sensor board 324 located in the distal housing 107. Controller board 310 communicates with controller circuit board 320, which communicates data and commands to a circuit board 322 and sensor board 324.

The circuit board 322 can include the camera, the camera chip and light source chip. The distal housing can be sealed 325 and is reusable using cleaning and sterilization techniques. The flexible section 105 can form a reusable endoscope component with the motorized section 103 and distal housing 107 or alternatively can be detached and disposed of after a single use.

Another preferred embodiment of a control system 350 is illustrated in FIG. 3C. In this embodiment, endoscope sections 101, 103 and 105 are disposable after a single use, whereas distal housing 107 and handle 352 are reused. In this embodiment, the motor control circuits for distal motors 360 are mounted on controller board 354 along with handle motor 356 controls and interface and haptic actuators 358. This reduces the cost of the disposable portions of the endoscope in this embodiment. Thus, after a given procedure, the disposable components are discarded or recycled, and a second disposable component is attached for a second procedure.

In one embodiment, the drive electronics for the endoscope system use H-bridges and an adjustable pulse width modulation (PMW) in order to obtain high instantaneous torques and for speed control of two degrees of motion. For example, the system can be controlled with a NI USP-6501 data acquisition system and L298 H-bridges. A Logitech® Attack™ 3 joystick and software written in NI LabVIEW 8.5, Visual Basic, or embedded C can be used to control the position of the tip of the endoscope. The endoscope can also be constructed with force sensors to measure and indicate the level of force applied to the tissue by the tip. The measured force can also be used to provide haptic feedback to the user through the handle to provide tactile sense of the level of force. The system can be programmed to limit the amount of force applied either by limiting the input to the motor or by feedback measurement of strain (where strain gages are placed along the length of the turning section 105) tip contact force (where sensors 465 are placed around the outside of 107), or through other methods. Position can be measured using two gyroscopes 312, 314 (one placed at the motor modules 103 and one placed at the tip 107 for a relative measurement), using strain gages (placed along the length of the turning section 105), using capacitive distance sensing (placing capacitive sensors 465 at the tip 107 or on any of the turning modules 105), using instruments to measure the string length (using potentiometers, encoders or other sensors to register the location of the motor shaft or string length placed at 103 or along 105), or through other methods. If the force is determined to be too high, the command to the motor can be reduced using negative feedback or the motor is driven to a safer position where less force is applied. To prevent perforations due to hooking, the tip can be straightened or driven to a safe position using the control system when the endoscope is withdrawn.

Various tools 475 can optionally be integrated into the distal housing 107 for manipulating devices for surgical procedures such as biopsy, resection, cautery or suturing.

A schematic illustration of a haptic feedback system 380 is shown in FIG. 3D. The user actuates the system using sensors 384 that operate a position control system 386, which in turn actuates 388 the endoscope 390. Position sensors 392 can provide feedback to control system 386 and force sensors 394 provide data to a resistance control system 396 which controls actuators 398 in the handle held by the user 382.

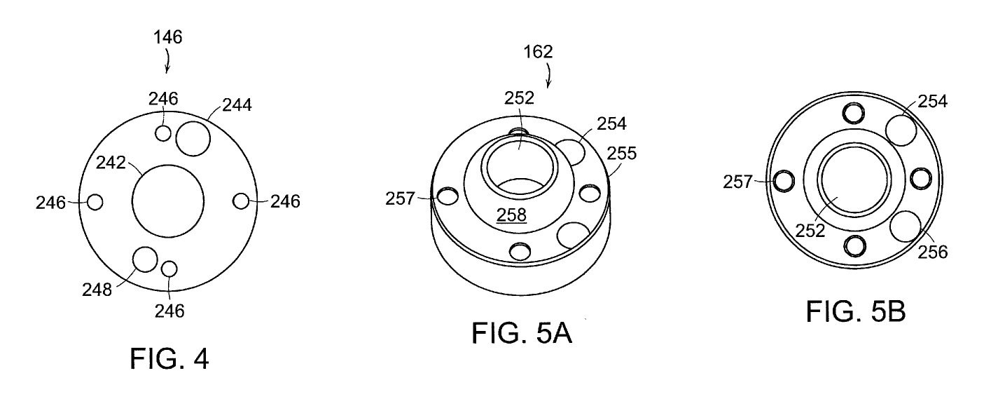

Illustrated in FIG. 4 is an individual turning module element 146. Each module 146 has a central opening 242, that can be about 4 mm in diameter, for example, a channel 244 for air, water or suctioning, four openings 246 for tip control cables, and hole 248 for electrical wiring to the distal end.

The body section 101 in the embodiment of FIG. 3A comprises a plurality of modules that are free to pivot relative to one another to provide flexibility to the body section 101. FIGS. 5A and 5B illustrate, respectively, a front perspective and a top elevation view of a body module 162 according to one embodiment of the invention. The body module 162 includes a central opening 252 extending through the module that forms and/or houses the inner lumen 25 of the endoscope 100. The module 162 also includes an opening 254 for passage of the air/water channel and an opening 256 for passage of wires, such as a motor control cable, that provide signals and power to components in the motor module 103 and tip module 107. In one embodiment, the central lumen opening 252 has a diameter of approximately 4 mm, the air/water channel 254 is approximately 2 mm in diameter, and the wire opening 256 is approximately 1.95 mm in diameter.

The body module 162 in this embodiment includes a generally disc-shaped, portion 255 having a first diameter and a spacer portion 258 having a second, smaller diameter. The spacer portion 258 extends in an axial direction from one surface of the disc-shaped portion 255. The central lumen opening extends through both the disk-shaped portion 255 and the spacer portion. The spacer portion is designed to provide spacing between the adjacent modules of the body section 101, and also facilitates the pivoting motion of the adjacent modules relative to one another. In the embodiment of FIGS. 5A and 5B, the spacer portion includes a generally convex curved outer surface. The curved outer surface of the spacer portion 258 allows the body module to easily pivot in any direction with respect to the adjacent body module. The spacer portion 258 is preferably integrally formed with the disc-shaped portion 255, though it will be understood that separate spacer elements can be provided between adjacent disc-shaped modules.

In the embodiment of FIGS. 5A and 5B, the body modules 250 have a plurality of small openings 257 to allow tensioning cables to pass through the body section 101. As shown in FIG. 3, the cables 208 are coupled to a motor in the handle 200 to enable adjustment of the body stiffness. This may be desirable, for example, during insertion of the endoscope into the patient's body, so that it can be easily inserted and advanced. As the length of the tensioning cables inside the body module section is increased, the amount of friction between adjacent body modules 162 is reduced thereby permitting the body modules to become increasingly free to pivot and move relative to one another, which increases the flexibility of the body section 101. The surface of the spacers 258 that contact the adjacent body module can have surface properties to enable the body to become rigid when sufficient tension is applied. This may be advantageous during removal of the endoscope from the body, so that it can be easily withdrawn from the body without causing damage and so that loops in the endoscope can be resolved.

In one embodiment, the motor 120 is a planetary geared motor, such as the GM15 pager motor from Solarbotics, Ltd. of Calgary, Alberta, which has a gear ratio of about 25:1 or higher. In one exemplary embodiment, the motor 120 has a diameter of approximately 6 mm, a length of about 20 mm, and a weight of about 1.2 grams. The motor in this embodiment is designed for 3 to 6 V operation, providing 1200 RPM while drawing 100 mA. The stall torque is over 20 g-cm.

The system is easily scaled to other sizes. Other motors can be used that have a sufficiently high output torque. Other suitable motors include several motors from Maxon Motor ag of Sachseln, Switzerland (www.maxonmotor.com), including, without limitation, Maxon model nos. 349190, 349191, 349192, 349189, 250101 and 310599, which can be combined with Maxon gear nos. 304179, 304180 and 304181.

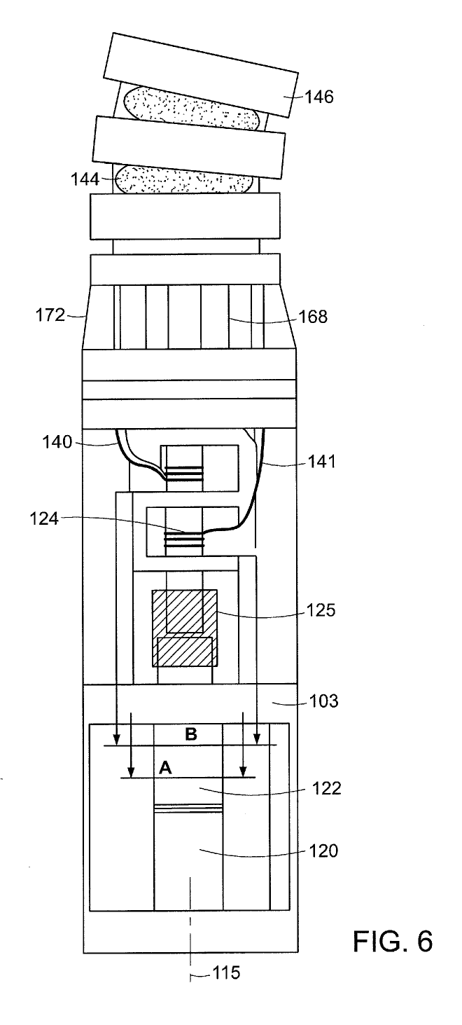

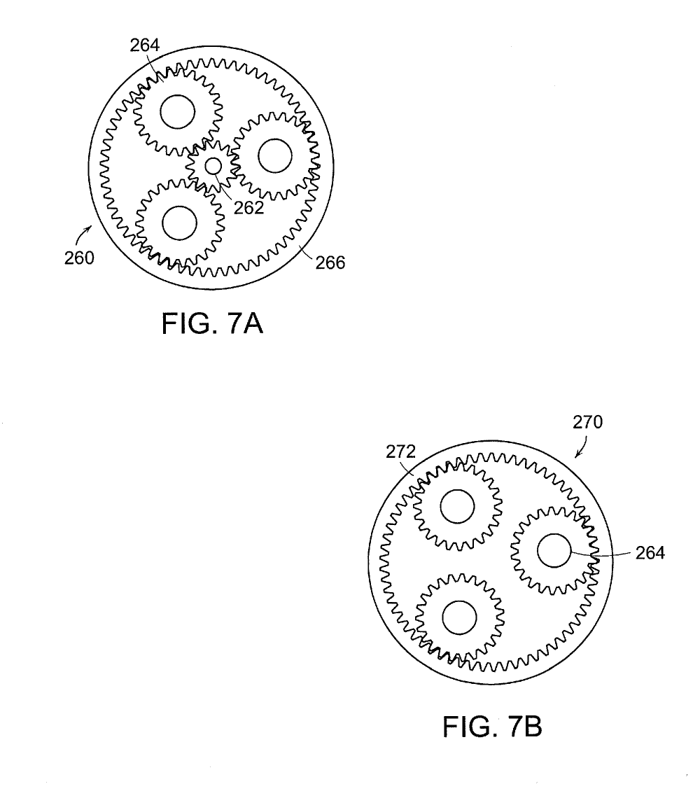

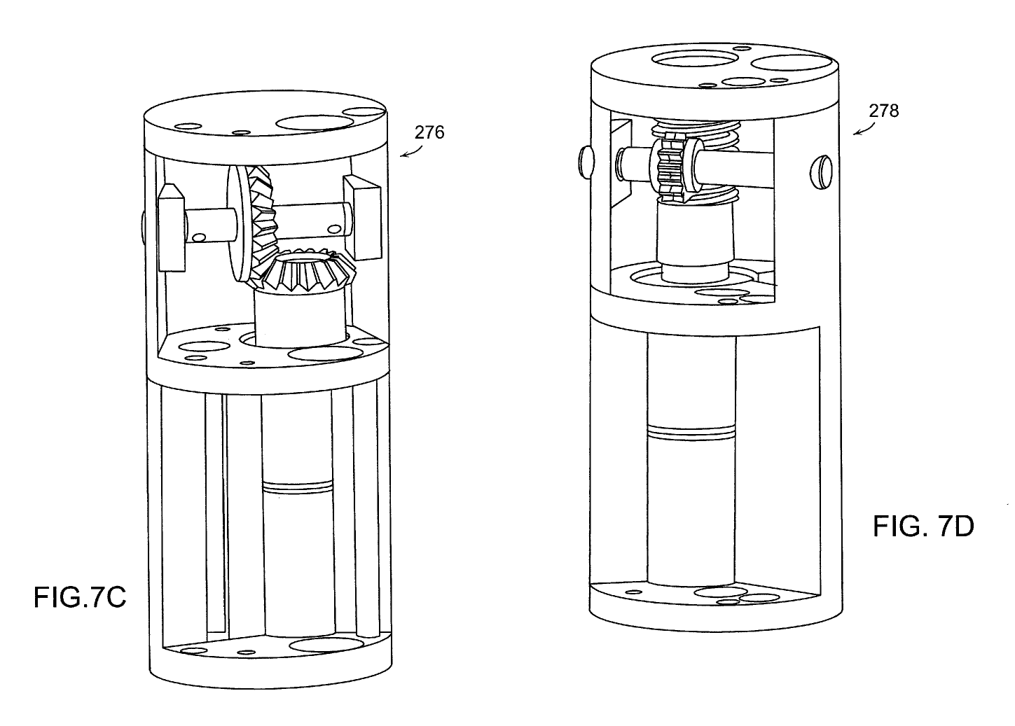

Illustrated in FIG. 6 is an enlarged view of the motor module 103. Note that a second motor can be mounted coaxially with the illustrated motor to provide directional control in two orthogonal planes extending through the longitudinal axis 115, thereby allowing the tip to deflect in any selected direction. A second motor can also be mounted in the same motor module next to the first motor. In FIG. 6, monofilament microwires or cables 140 and 141 are attached to shaft 124 that is connected to the gear assembly 122 with a coupler 125. As the shaft rotates in one direction, one of the cables 140, 141 unwraps from the shaft while the other wraps around the shaft by an equal amount, thereby displacing the tip. The wires from the first motor 170 and second motor 171 (FIG. 3) extend through converter 168 in an orthogonal plane to that occupied by wires 140, 141. The planes A and B in FIG. 6 define the sectional views of the gear assembly 122 shown in FIGS. 7A and 7B, respectively, the sungear 262, driven by the motor 120, engages the planetary gears 264 within annular gear 266. As seen in FIG. 7B, the gears 264 drive the output gear 272 coupled to the shaft 124. Alternate gearing configurations with Miter gears 276 or worm gear 278 driving shafts can also be used as shown in FIGS. 70 and 7B, respectively, to increase output torque. Such gear systems can also be used to control tools in the distal housing 107.

The safety and holding modules can be implemented in several ways. The safety modules and holding modules can be actuated using a pull strings (as shown with the cables or strings 209 in FIG. 3A) attached to blades located in 108 for the safety module and clamps located in 109 for the holding module. The safety module can also be actuated using a motor module coupled with blades that cut the wires when commanded. Other methods using pressure, melting, or vibrational cutting can also be used.

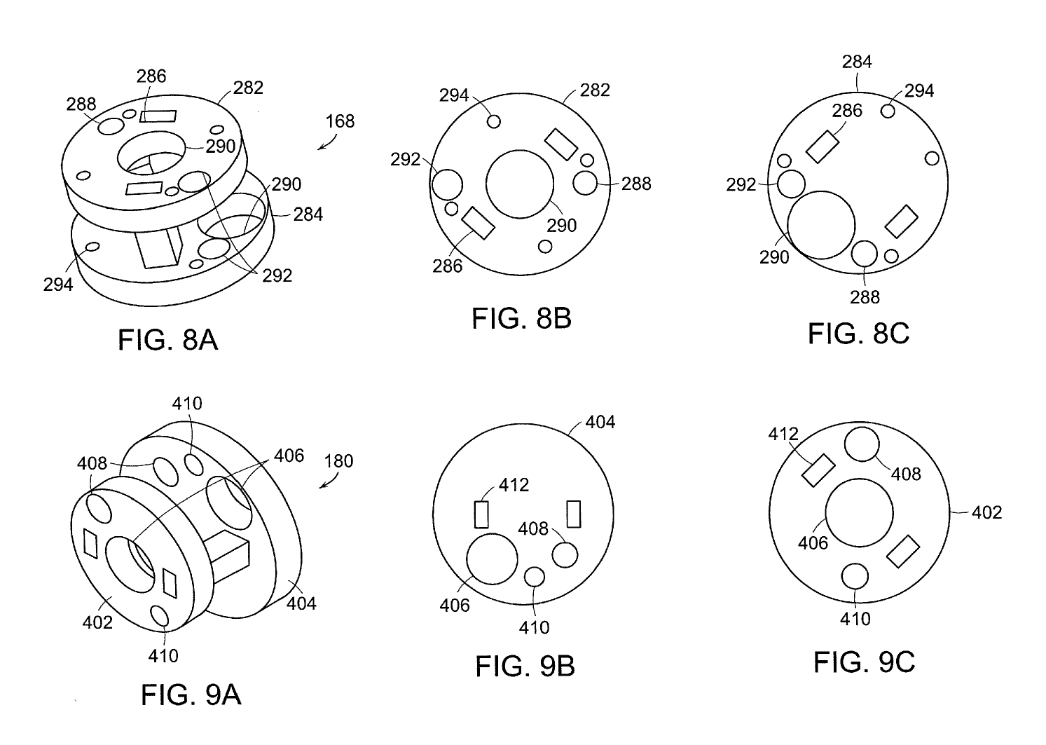

As shown in FIGS. 8A-8C, a motor converter 168 has a first disc 282 connected to a second disc 284 with converter bars 286. The central lumen 290 (accommodating central lumen 25) passes through the center of disc 282 and is offset from the center on the second disc 284. The channels 292 for air, water and suctioning are aligned. The holes for electrical wiring 288 and holes 294 for cables 140, 141 are also shown. The offset configuration accommodates bending to one side.

Shown in FIGS. 9A-9C is a preferred embodiment of a converter 180 from the flexible section to the distal housing 107. A first disc 402 is connected to second disc 404 by converter bars 412. The air, water or suction holes 408 are aligned. The central lumen opening 406 in disc 402 is centrally positioned and is offset in disc 404. The wiring passage 410 can also be offset through the two panels.

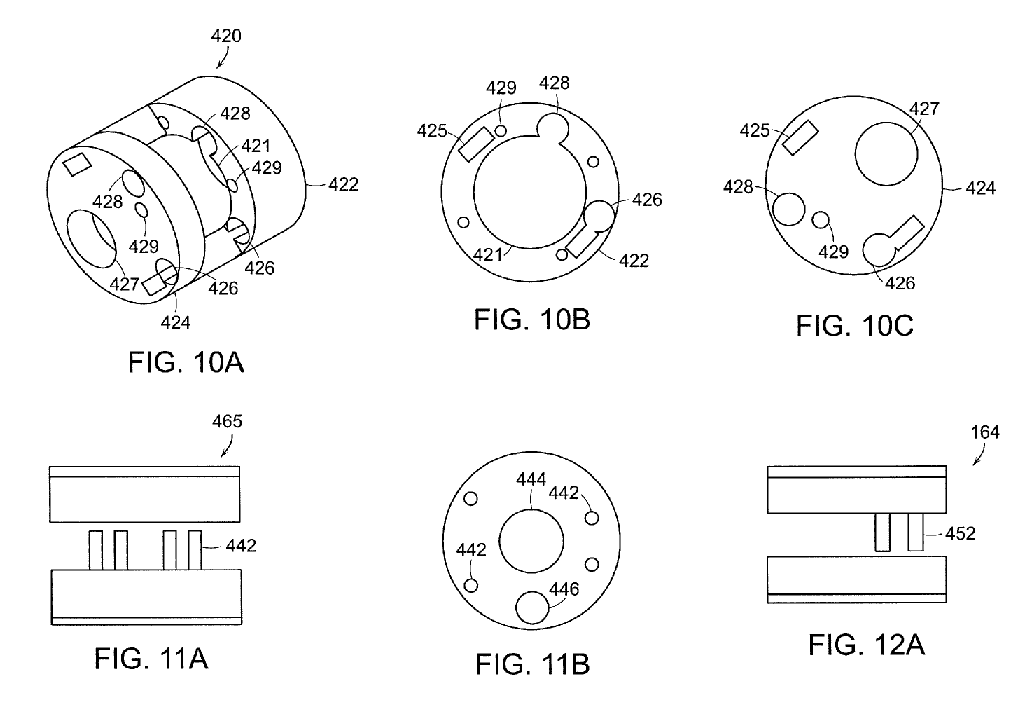

FIGS. 10A-10C illustrate a body to handle converter 420 has a first disc 424 that is connected to the handle and a second disc 422 connected to the body section 101. Holes 429 for tensioning cables 208, for air, water or suction 428, and for electrical wiring 426 are shown. The central lumen 427 of disc 424 is offset, while the central lumen opening 421 in disc 422 is enlarged.

FIGS. 11A and 11B show a preferred embodiment of a connector 165 to the tip with pins 442 connecting elements with opening for air, water or suction 446 and a central lumen 444. Note that converter and connector functions can be integrated into a single holding module.

FIGS. 12A and 12B illustrate side and sectional views of connector 164 of the body section 101 to the motor module 103 with pins 452 connecting discs with holes for the central lumen 456 and air, water (fluid) or suction 454.

A preferred embodiment of a distal housing 107 is shown in the perspective and end views of FIGS. 13A and 13B, respectively. The distal opening 470 of the central lumen and for air, water (fluid) or suction 468 is offset. The camera 462 can be positioned between LEDs 464 and 466. The housing 107 can also include a circuit board assembly for the camera and LEDs, as well as other components.

The force that the tip of the endoscope system can produce determines the maneuverability of the system. However, the tip force should not exceed the perforation force of healthy tissue (in the case of operator error). Therefore, a model can be used to guide the selection of the geared motor and articulated tip dimensions and force profile.

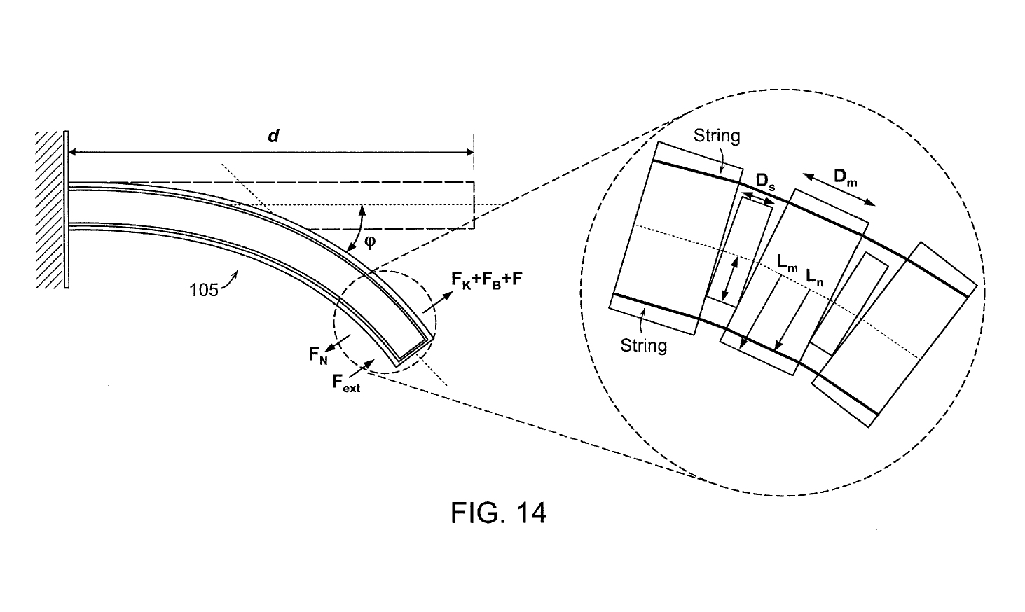

FIG. 14 illustrates the geometry of a steerable endoscope for modeling the angle to tip force relationship. The maximum bending angle is defined by the relative sizes of the tip modules and spacers. For n spacers with length Dₛ and length Lₛ and module radius of Lₘ, a first order estimate of the maximum bending angle can be obtained,

$${ϕ}_{max} = 2 n {sin}^{- 1} ( \frac{{D}_{s}}{2 ( {L}_{m} - {L}_{s}} ) . \qquad ( 1 )$$

By balancing forces at the tip of the endoscope in the normal direction, it is possible to obtain Equation 2. The forces, in order, are the externally applied normal force (which is measured), the force associated with the spring constant, the force associated with damping, the force associated with inertia, and the internal normal force from cable tension. Results from other configurations with additional contact forces can be derived similarly.

Fₑₓₜ+Fₖ+F_{B}+F_{J}−F_{N}=0 (2)

From measured data, it was found that the spring force is Fₖ=Kφ, the damping force is F_{B}=B\dot{φ} and the inertial force is F_{J}=J\ddot{φ}. Note that Equation 2 only exists for positive values of F_{N} Fₑₓₜ and since the cable pulling procedure produces force in only one direction. The internal normal force from cable tension is related to the tangential string tension and this relation can be derived with a torque balance for the tip module at the very end. The tip force can be approximated using the capstan friction equation where μ is the coefficient of friction, Fᵢₙ is the force output of the motor, and Fₜᵢₚ is the force measured at the tip. An extra π/2 term occurs due to the capstan friction in the 90° turn inside the rotary to linear transmission. This is shown in Equation 3.

$${F}_{N} = \frac{{L}_{h} - {L}_{s}}{{D}_{m} + {D}_{s}} {F}_{tip} = \frac{{L}_{h} - {L}_{s}}{{D}_{m} + {D}_{s}} {F}_{in} {e}^{- μ ( ϕ + \frac{π}{2} )} \qquad ( 3 )$$

The input force can be measured or calculated using the input voltage V, gear ratio N, motor constants Kₑ, and Kₜ, radius of the motor shaft r, resistance of the motor R, and angle of the motor shaft θ. Equation 4 shows this relation:

$${F}_{in} = {NK}_{t} \frac{V - {K}_{e} \dot{θ} / N}{R r} . \qquad ( 4 )$$

The full dynamics can be obtained in Equation 5:

$${F}_{ext} = \frac{{L}_{h} - {L}_{s}}{{D}_{m} + {D}_{s}} ( {NK}_{t} \frac{V - {K}_{e} \dot{θ} / N}{R r} ) {e}^{- μ ( ϕ + \frac{π}{2} )} - K ϕ - B \dot{ϕ} - J \ddot{ϕ} . \qquad ( 5 )$$

A relationship between the motor angle and the bending angle can be derived from geometry. The relationship shown in Equation 6 is valid up to φₘₐₓ. The approximation on the right can be used for small φ/n,

$$θ = \frac{n ( {L}_{h} - {L}_{s} )}{r} sin ( ϕ / n ) \approx \frac{( {L}_{h} - {L}_{s} )}{r} ϕ . \qquad ( 6 )$$

For a static system, Equation 5 reduces to Equation 7 for an input torque of Tᵢₙ:

$${F}_{ext} = \frac{{L}_{h} - {L}_{s}}{{D}_{m} + {D}_{s}} \frac{{T}_{in}}{r} {e}^{- μ ( ϕ + \frac{π}{2} )} - K ϕ . \qquad ( 7 )$$

The spring constant K is a function of the elasticity of the endoscope tip (including the tool channel material, wire and tubing), the moment of inertia, and the length of the endoscope tip, d. The measured data can be used select the desired force and speed characteristics of the system.

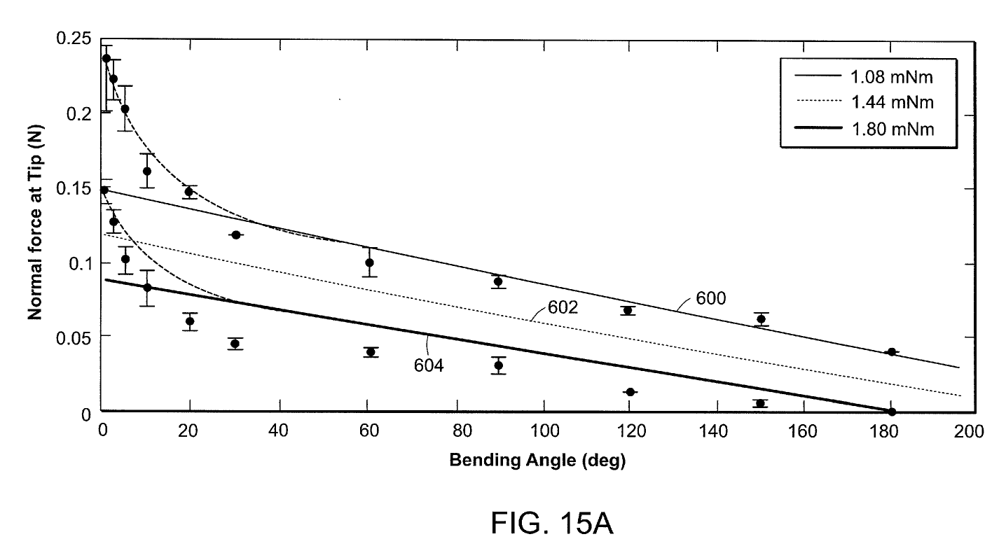

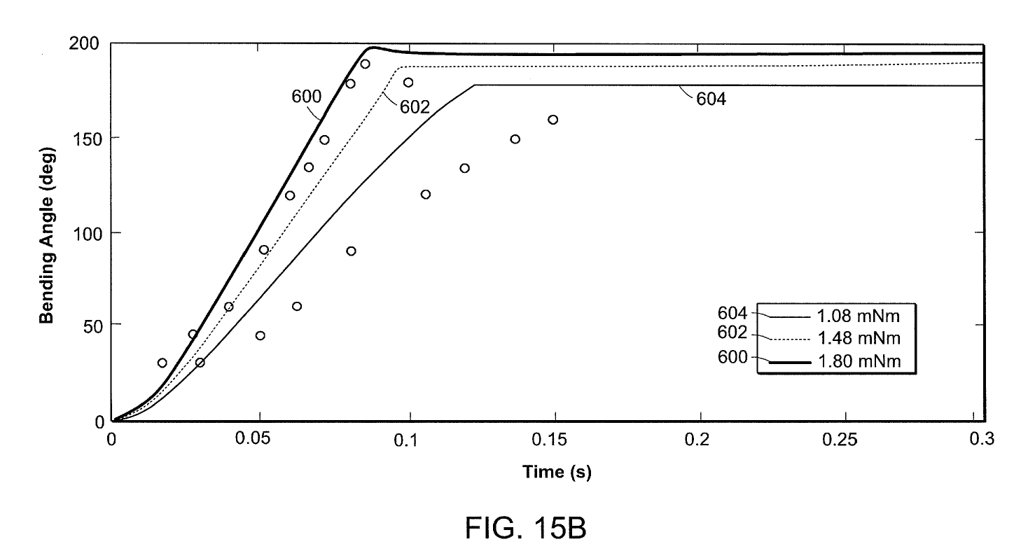

The forces exerted by the endoscope tip were measured by holding the motor module fixed and measuring the force against the tip at different bending angles. The results of the measurements along with the analytical representation for the static tip force are plotted on FIG. 15A, which shows the static normal forces at the tip as a function of the bending angle for different input torques. The dots with standard deviation bars represent measured data, the solid lines represent model predictions for selected limits, and the dotted lines represent model predictions for selected limits with a low angle correction.

The measured spring constant in this example is 0.020 N/rad, the measured coefficient of static friction is 0.28 rad⁻¹, and the measured coefficient of sliding friction is 0.10 rad⁻¹. The data with standard deviation bars matches well with the predicted output for larger angles, but a low angle correction in the form 1+C₁exp(−C₂φ) is desirable for smaller angles. At low angles, the cable tension has more leverage than predicted by the representation because the play in the hole for the cable allows the selection of different bending points. The correction factor, which represents the exponentially decreasing leverage from cable tension, is multiplied with the tip force. In FIGS. 15A and 15B, the line 600 corresponds to 1.80 mNm for the input torque, line 602 corresponds to 1.44 mNm for the input torque and 604 corresponds to 1.08 mNm of input torque.

In addition to the static tip forces, the dynamics of the endoscope can also be plotted using the dynamic model with Fₑₓₜ=0, and this is shown in FIG. 15B, which shows the bending angle as a function of time for different input torques. The dots represent measured data and the lines represent model fits. The dimensions were chosen based on iterative design with 6 mm motors: n=11, r=1.1 mm, Dₛ=0.7 mm, Dₘ=3.1 mm, Lₛ=3.3 mm, Lₘ=5.5 mm, and Lₕ=3.7 mm. The results correlate with data from high-speed camera images at 600 fps (representative points plotted) taken of the system at different input torques associated with different input voltages. As the input torque increases, the endoscope tip approaches the maximum bending angle more quickly. If the motor voltage is too low, the endoscope is incapable of reaching the maximum angle. When the endoscope tip reaches the angle limit, the tip can oscillate slightly by deforming in directions out the plane of cable actuation. The fitted values for the dynamic model are J=9.0×10⁻⁵ N/(s²·rad) and B=1.5×10⁻³N/(s·rad).

In order to assess the system, the tip forces are compared to the critical contact pressures of human organs, which are on the order of 20 kPa. This indicates that the resistive force of the organ is at minimum 0.64 N for a tip module of 11 mm in diameter and 2.9 mm in length. The system design produces forces well below this value and system parameters can be selected to prevent forces from reaching critical contact pressures in possible failure modes. Conventional endoscope designs with longer cable actuation mechanisms are susceptible to additional forces from bends in the body of the endoscope. The motor torques for these systems are much higher and the operator is more likely to produce tip forces that could potentially puncture organs. Thus, for each application, such as for the colon or other regions of the gastrointestinal system, the maximum force exerted by the distal end of the endoscope is maintained below a threshold force level defined by the perforation force for the type and condition of the tissue. Such force levels can be programmed into the computer for selection by the user thereby utilizing a plurality of programmable force levels that are stored in memory.

Having described the above illustrative embodiments of the presently disclosed tip actuated disposable endoscope, other alternative embodiments or variations may be made. For example, endoscopes are widely used for diagnostic purposes, but endoscopic procedures typically involve biopsies to remove tissue for further examination. Such endoscopes are operative to control the motion of not only the endoscope itself, but also any tools that are inserted within it for these types of procedures. Because biopsy tools are generally stiffer than the endoscope, such endoscopes can account for this stiffening when implementing an actuation method for turning.

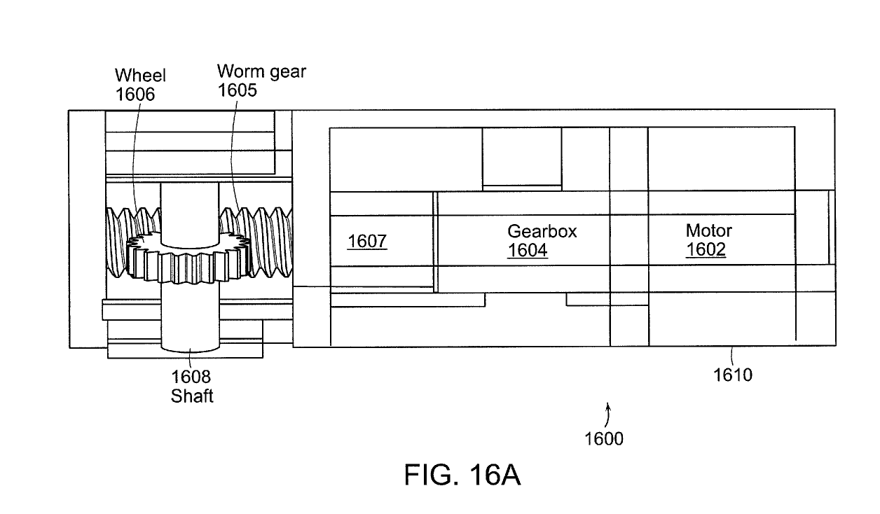

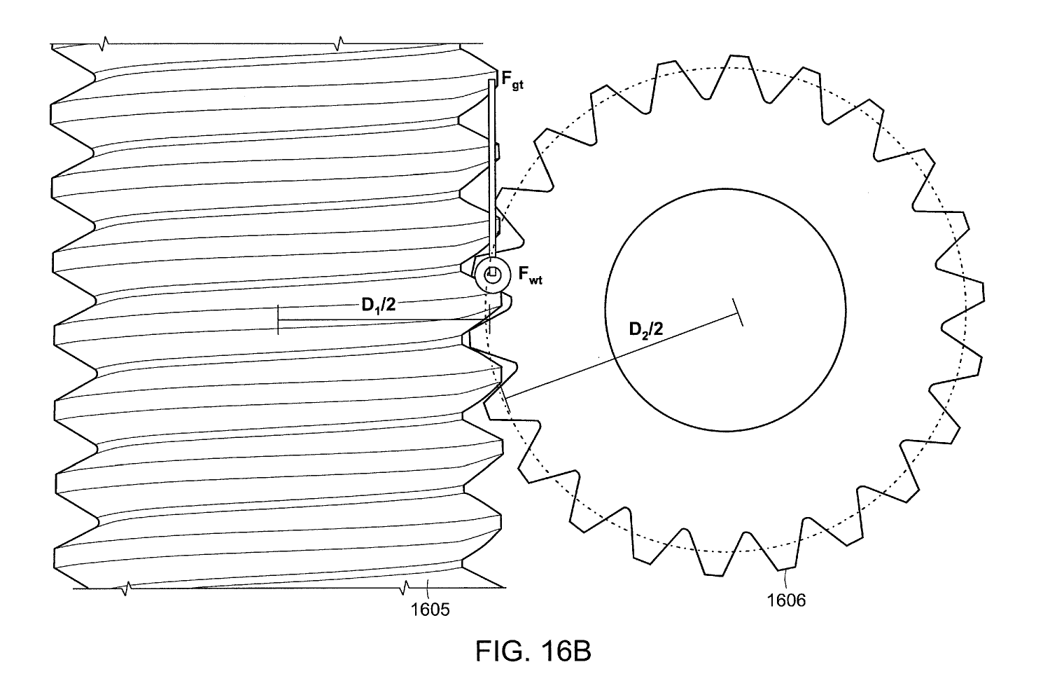

FIG. 16A depicts an illustrative alternative embodiment of a motor module section 1600 for such endoscopes that can be employed to increase the torque to turn an inserted biopsy tool with the rest of the endoscope. As shown in FIG. 16A, the motor module section 1600 includes a motor 1602, a gearbox 1604, a worm gear 1605, a wheel 1606, and a shaft 1608. For example, the motor 1602 may be a DC motor or any other suitable type of motor, and the gearbox 1604 may be a 25:1 gearbox or any other suitable gearbox. The motor module section 1600 is operative to increase a pulling force to ease the bending of a stiffened scope.

As further shown in FIG. 16A, an output shaft 1607 of the motor 1602 and the gearbox 1604 is coupled to the worm gear 1605, which drives a worm wheel 1606, and causes rotation of the shaft 1608. For example, the reduction of the worm assembly including the worm gear 1605 and the wheel 1606 can be 23:1, or any other suitable ratio. In accordance with the illustrative embodiment of FIG. 16A, the gear train results in a total 575:1 gear reduction. Such a reduction can supply sufficient torque to pull a stiffened scope and a biopsy tool. A stiff motor module 1610 surrounds and holds the motor 1602, and bears the worm gear 1605. It is noted that brass bearings can be added for the tip of the worm gear 1605 and the shaft 1608, and that E-clips can be used to constrain the shaft 1608 to rotation only.

The following analysis is provided to determine, a suitable gear efficiency of the worm assembly including the worm gear 1605 and the wheel 1606 (see FIG. 16A). The tangential force on a worm gear, F_{wt}, can be calculated from a torque equation by dividing the worm torque by the pitch radius, as follows,

$${F}_{wt} = \frac{2 {M}_{1}}{{D}_{1}} \qquad ( 8 )$$

in which M₁ is the worm torque, and D₁ is the pitch diameter of the worm gear (see also FIG. 16B). Using equation (8) above, the tangential force on the worm wheel 1606, F_{gt}, can be calculated, as follows,

$${F}_{gt} = {F}_{wt} [ \frac{cos {∝}_{n} - μ tan γ}{cos {∝}_{n} tan γ + μ} ] \qquad ( 9 )$$

in which μ is the coefficient of friction, αₙ is the normal pressure angle (in degrees), and γ is the worm lead angle (in degrees). For example, αₙ is can be 20 degrees or any other suitable value.

Using equation (9) above, the output torque, M₂, can be determined by multiplying the pitch radius of the worm wheel 1606 by the tangential force of the worm wheel 1606, as follows,

$${M}_{2} = {F}_{gt} \frac{{D}_{2}}{2} \qquad ( 10 )$$

in which D₂ is the pitch diameter of the worm wheel 1606 (see also FIG. 16B).

It is noted that the efficiency of the worm assembly can range from about 20% to close to 100% based on the geometry of the worm gear 1605 and the wheel 1606. The efficiency, E, can be calculated by dividing the output torque with friction by the output torque without friction, as follows.

$$E = \frac{cos {α}_{n} - μ tan γ}{cos {α}_{n} + μ cot γ} \qquad ( 11 )$$

The normal pressure angle, αₙ, for worm gears is typically 20 degrees, given low coefficients of friction. To optimize the efficiency, E, the worm lead angle, γ, can therefore be between 40 and 50 degrees. Accordingly, assuming a coefficient of friction value of about 0.3, the calculated efficiency, E, for the worm gear 1605 is about 56%.

It is further noted that materials that can be used to reduce friction include hardened steel for the worm gear 1605, and phosphor bronze for the worm wheel 1606. Such materials are typically strong enough such that the gear teeth will not fail under load. In accordance with an exemplary embodiment, the worm wheel 1606 and the worm gear 1605 can be made from hardened steel and 4042 steel, respectively. For example, the worm gear 1605 can be cut on an electric discharge machine. Further, the bearings for the motor module section 1600 can be made of brass, and can be encased by Acura 40 stereolythography material. There can also be a tool passage of about 4 mm in diameter within the casing. Such a case can be injection molded to reduce costs. The motor 1602 can be a low-cost gear head motor with a diameter of about 6 mm. The motor module 1610 can be a maximum of 14 mm in diameter, but can be scaled smaller as manufacturing methods are improved.

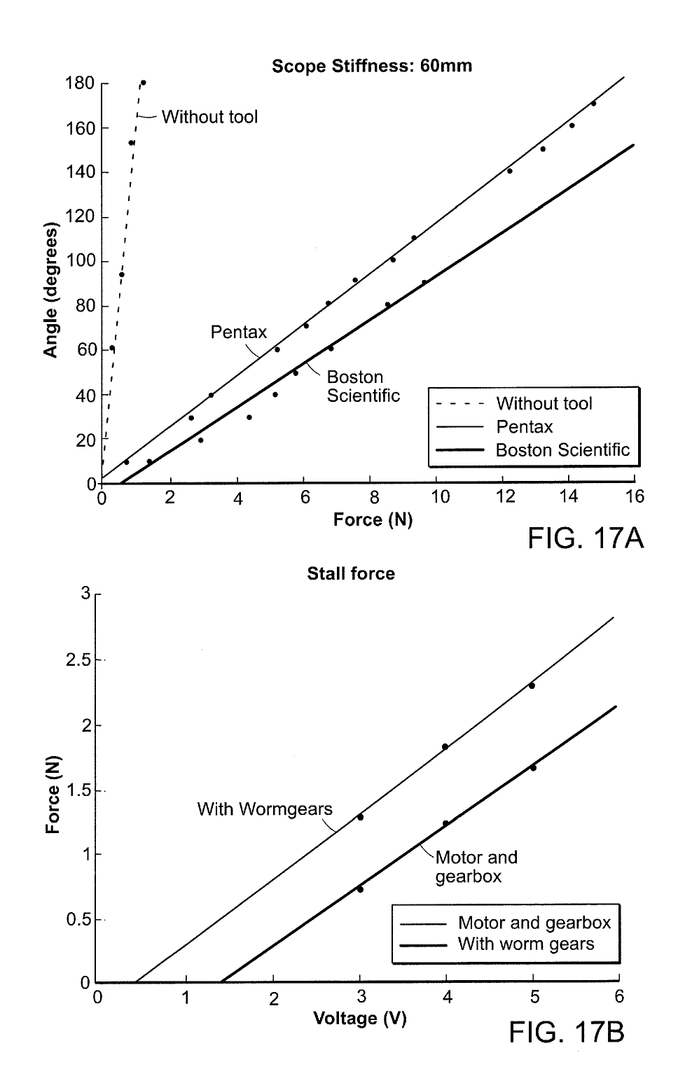

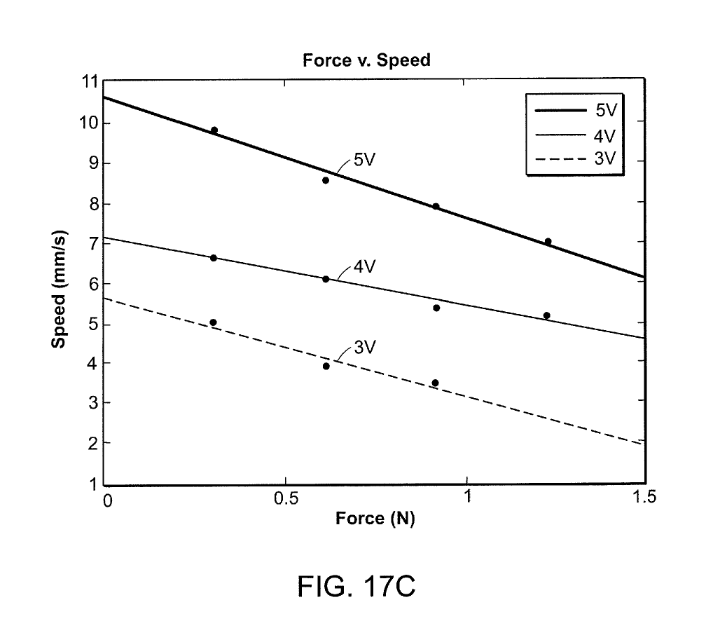

In accordance with another exemplary embodiment, the stiffness of the turning section of an endoscope incorporating the motor module section 1600 (see FIG. 16Aa) was measured, without a biopsy tool, as well as with two different biopsy tools (Pentax, Boston Scientific). In this exemplary embodiment, the length of the endoscope was about 60 mm. The measurement results, with and without the biopsy tools, are plotted in FIG. 17A. It is noted that without the biopsy tool, the force needed to turn the endoscope 180 degrees is significantly less than when a biopsy tool is inserted; hence the need for the worm assembly. It is further noted that the Pentax forceps, which are made of a flexible coiled spring, are less stiff than the Boston Scientific forceps, which are mostly made of a stiffer plastic.

In further accordance with this exemplary embodiment, stall force and force versus speed characteristics were measured, focusing on the motor module section 1600 and the performance of the worm assembly. The output stall force (measured as a pulling force on the monofilament cables) as a function of voltage is plotted in FIG. 17B. As shown in FIG. 17B, the addition of the worm assembly shows an increase in the stall force over the motor and gearbox alone. Further, the output speed as a function of pulling force for various voltages are plotted in FIG. 17C. It is noted that the speed per unit force is lower with additional worm gears than with the gearbox and motor alone. The speed per unit force also decreases with decreasing voltage.

In addition, endoscopic procedures such as colonoscopies, natural orifice translumenal endoscopic surgery, minimally invasive pericardioscopy, etc., can benefit from the present endoscope having features such as assisted tool positioning, closed loop position control, feature tracking and image stabilization, built-in force limits, and haptic feedback. Further, cauterization, ablation, and biopsy instruments are some exemplary tools used in conjunction with preferred embodiments of the present endoscope. When performing endoscopic procedures, medical professionals typically position tools, along with an endoscope camera, at several locations of interest. Using an endoscope with closed loop position control, feature tracking, and/or image stabilization, a user can be free to manipulate other tools, while the endoscope maintains the view of the camera or the location of one of the tools, thereby lessening the cost and reducing the complexity of such procedures.

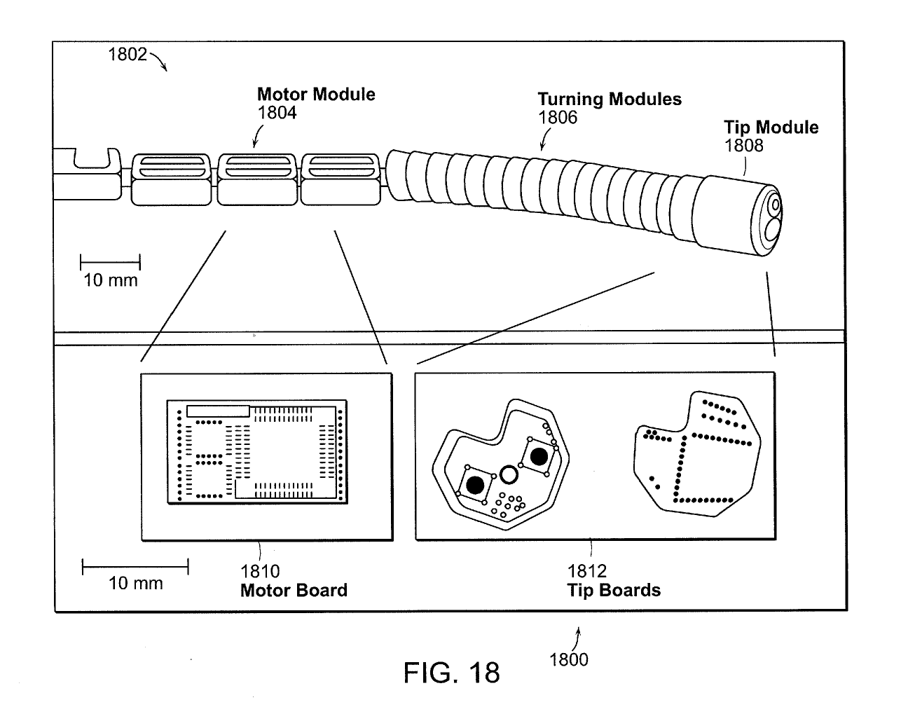

FIG. 18 depicts an illustrative alternative embodiment of an architecture for an endoscopic robotic platform 1800 that incorporates such features. As shown in FIG. 18, the endoscopic robotic platform 1800 includes an endoscope 1802 having a motor module 1804, a plurality of turning modules 1806, and a tip module 1808. The motor module 1804 includes a motor board 1810 having a microprocessor, a gyroscope, and motor drivers such as rotary motor drivers. The tip module 1808 includes a plurality of tip boards 1812 having a camera, a gyroscope, a microprocessor, and multiple lights.

The endoscopic robotic platform 1800 of FIG. 18 is modular so that different modules can be easily swapped to achieve higher pulling forces, using geared rotary-to-linear transmissions. Such a distributed modular architecture has several benefits. First, the modular architecture provides distributed computing resources, which enables dedicated local calculations for image acquisition and sensor integration. Second, the modular architecture reduces the number of wires that need to be passed through the body of the endoscope 1802, thereby reducing the stiffness of the overall system. The modular architecture also allows for a simpler interface between single-use components and permanent components.

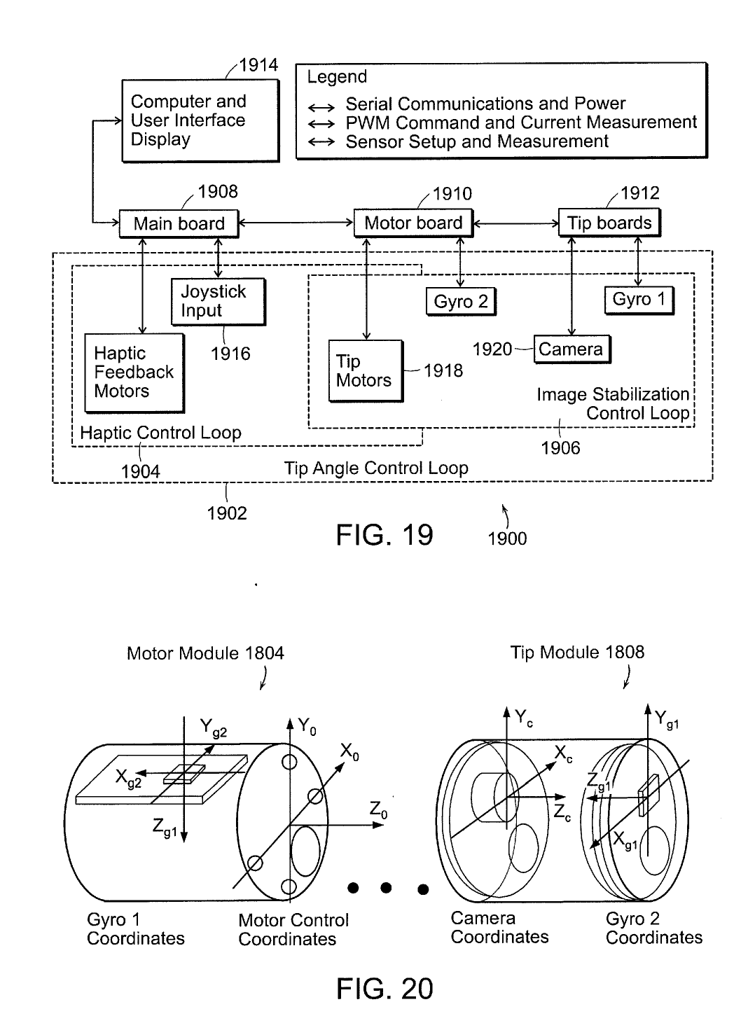

FIG. 19 depicts an exemplary sensor and actuator architecture 1900 for the endoscopic robotic platform 1800 of FIG. 18. As shown in FIG. 19, the sensor and actuator architecture 1900 includes a tip angle control loop 1902, a haptic control loop 1904, an image stabilization control loop 1906, a main board 1908, a motor board 1910, several tip boards 1912, and a computer and user interface display 1914. The sensor and actuator architecture 1900 includes electronics that are located inside the single-use endoscope 1802, and more complex electronics that are located in a permanent housing, which is attached to the computer and user interface display 1914. The main board 1908 includes permanent electronics such as a user input joystick and haptic feedback motors, as well as a microprocessor, such as an Arm Cortex M3 microprocessor, which can interface with the computer 1914 using USB. To keep the overall layout modular, the main board 1908 can interface with the endoscope electronics via a synchronous serial peripheral interface bus.

It is noted that the motor board 1910, which lays horizontally along the motor module 1804, houses a smaller form factor microprocessor, such as an Arm Cortex M3 microprocessor, which commands motor drivers in PWM mode, and measures motor current to control two actuation motors. The motor board 1910 also houses a three-axis gyroscope (“Gyro 2”), such as an InvenSense ITG-3200 gyroscope, which is used to determine the turning angle of the endoscope tip. The motor board 1910 can interface with the tip boards 1912 via a second synchronous serial peripheral interface bus. The tip boards 1912 include a VGA camera, such as a Toshiba TCM8230MD camera, which can communicate through 8-wire parallel lines with a smaller form factor microprocessor, such as an Arm Cortex M3 microprocessor. The tip boards 1912 also have an extension board with two white LED lights with luminous power of, for example, up to 120 lm.

The tip boards 1912, which can be under 12 mm in diameter and can contain a notch or lumen for a tool passage, house a three-axis gyroscope (“Gyro 1”). The two gyroscopes Gyro 1, Gyro 2 are operative to determine the relative angle between the motor module 1804 and the tip module 1808. The gyroscopes Gyro 1, Gyro 2 can be used to implement accurate blind turns (i.e., turns that may be outside the field of view of the camera), and to provide higher speed responses to angle perturbations than the camera and an image processing algorithm are able to output. Because of possible drift, the gyroscope integration and calibration can be done on a local board, and the gyroscopes can be used for relative turns.

FIG. 19 illustrates the three control loops 1902, 1904, 1906 that are used to drive the system. The system enables turning speeds of 400 degrees per second or more. The outer tip angle control loop 1902 includes all of the sensors and actuators. This control loop 1902 takes inputs from a joystick 1916, and sends angle commands to tip motors 1918 using the gyroscopes Gyro 1, Gyro 2 for accurate closed loop turns. In this loop 1902, a user can utilize a camera 1920 to determine positioning, or can request blind turns. The next loop is the image stabilization control loop 1906, which includes the gyroscopes Gyro 1, Gyro 2, the camera 1920, and the tip motors 1918. This loop 1906 processes the images from the camera 1920 to determine the movements necessary to keep an object of interest in the center of the image. The gyroscope readings are used for the closed loop control, and the camera information is used as the input command. The third control loop 1904 is for the haptic system, which measures the motor output torque and translates it to a resistance on the joystick 1916 to indicate to the user the amount of force being applied at the endoscope tip. It is noted that the sensor interfaces and control algorithms for the sensor and actuator architecture 1900 can be written in embedded C and assembly language.

The modular motorized design of the endoscopic robotic platform 1800 has several features for controls that involve inaccessible angular locations, nonlinearities in the drive dynamics, and windup delay when reversing inputs. For example, with one motor pulling in the x-axis of a coordinate system, and another motor pulling in the y-axis of the coordinate system, it may be impossible to access all possible angles if the x and y-axes are limited to 180 degrees. In addition, a single motor per axis with a linear-to-rotary transmission generally has one taut cable and one loose cable, which can cause windup delay when reversing directions. Further, the friction and backlash in the gearing may cause a dead-band at low input voltages. For example, the dead-band nonlinearity, and the input limit nonlinearity in the x (or y) direction can be modeled, as follows,

$${V}_{inz} = \{ \begin{matrix}sgn ( {V}_{x} ) {V}_{max} & if \vert {V}_{x} \vert < {V}_{max} , \\ {V}_{x} & if \vert {V}_{x} \vert \geq {V}_{min} , \\ 0 & otherwise ,\end{matrix} \qquad ( 12 )$$

in which Vₓ is the input voltage, Vₘᵢₙ is the minimum voltage necessary to overcome static frictional losses in the system, and Vₘₐₓ is the maximum input voltage to the motor.

The windup delay nonlinearity can also be modeled. If an input command is proceeding in one direction (grouped by Vᵢₙₓ>0 or Vᵢₙₓ<0), then the dynamic equation for angular movement in a single axis can be derived from a force balance, a torque balance, and by accounting for capstan friction. The windup delay nonlinearity can therefore be modeled (following earlier variable conventions except the angle is now denoted by the variable X),

$$J \ddot{x} + B \dot{x} + Kx - \frac{{L}_{h} - Ls}{r ( {D}_{n} + {D}_{s} )} {T}_{in} {e}^{- μ ( x + \frac{π}{2} )} = 0 , \\ {T}_{in} = \frac{N {K}_{t}}{R} ( {V}_{inx} - \frac{{K}_{e} n ( {L}_{h} - {L}_{s} )}{rN n} cos ( x / n ) \dot{x} ) , \qquad ( 13 )$$

in which J, B, and K describe the inertial, damping, and spring constants, respectively, Lₕ is the distance of a pull string from the center of the turning modules 1806, Lₛ is the radius of the spacer between the turning modules 1806, Dₘ is the height of the turning modules 1806, Dₛ is the height of the spacer, r is the radius of the torque transmission shaft, and μ is the coefficient of friction. Further, the maximum angular limit is

$${x}_{max} = 2 n {sin}^{- 1} ( \frac{{D}_{s}}{2 ( {L}_{m} - {L}_{s} )} ) \qquad ( 14 )$$

in which “Lₘ” is the radius of the turning modules 1806. The input torque, Tin, is a function of R, which is the resistance of the motor, Kₜ and Kₑ, which are the motor constants, N, which is the motor gear ratio, and n, which is the number of turning modules 1806. While one cable of length, sₓ₁, is taut, the opposing cable of length, sₓ₂, becomes looser, as follows,

sₓ₁=d−Lₕx−nK_{c}Dₛx,

sₓ₂=d+Lₕx+nK_{c}Dₛx, (15)

in which d is the length of the turning module section, and K_{c} is the compression of each of the spacers. When the motor changes directions (e.g., from Vᵢₙₓ>0 to Vᵢₙₓ<0, or vice versa), the motor winds up the loose cable, the tip angle is preserved, and all derivatives are set to zero (x=x, \dot{x}=0, \ddot{x}=0). The wind up speed is a function of input voltage, and can be expressed as

$$\frac{d {s}_{x}}{d t} = \frac{rN}{{K}_{e}} {V}_{inx} . \qquad ( 16 )$$

In the event that the cable is taut (sₓ=d+Lₕₓ), the single sided dynamic equations (see equations (13) above) can be solved. The equations (13) can then be used to predict the behavior of the robotic endoscope, and to guide the controller design.

To translate sensor readings into control inputs, the sensor information can be translated in a given sensor's frame of reference to the motor control frame of reference. FIG. 20 depicts the nominal alignment of each sensor relative to the motor module control coordinate system. Each of these coordinate systems is translated back to the motor control frame of reference, and mapped to an appropriate sensor reading in the x-rotational frame and the y-rotational frame, which correspond to the two turning motors, as follows,

$${x}_{i}^{*} = cos ( {β}_{i} ) cos ( {γ}_{i} ) {x}_{i} - cos ( {β}_{i} ) sin ( {γ}_{i} ) {y}_{i} + sin ( {β}_{i} ) {z}_{i} , \\ {y}_{i}^{*} = [ cos ( {α}_{i} ) sin ( {γ}_{i} ) + sin ( {α}_{i} ) sin ( {β}_{i } ) cos ( {γ}_{i} ) ] {x}_{i} + [ cos ( {α}_{i} ) cos ( {γ}_{i} ) - sin ( {α}_{i} ) sin ( {β}_{i} ) sin ( {γ}_{i } ) ] {y}_{i} - sin ( {α}_{i} ) cos ( {β}_{i } ) {z}_{i} , \qquad ( 17 )$$

in which xᵢ, yᵢ, and zᵢ are the rotational rate outputs (in radians/s) or integrated rotational angle outputs (in radians) of the sensors, and x*ᵢ and y*ᵢ are the desired readings used by the controller. The counterclockwise rotation angles α, β, and γ along the x, y, and z axes, respectively, are nominally α_{g1}=0, β_{g1}=π, and γ_{g1}=0 for the tip gyroscope (Gyro 1), and α_{g2}=0, β_{g2}=−π/2, and γ_{g2}=π/2 for the gyroscope (Gyro 2) in the motor module 1804 when calibrated to zero, while the motor module 1804 and the tip module 1808 are aligned. For the camera 1920, the rotational outputs for tracking objects, or for tracking relative motion, can be approximated for small rotations, as follows,

$$Δ {x}_{c} = \frac{{p}_{x}}{{t}_{x}} {f}_{x} \qquad ( 18 )$$

in which pₓ is the displacement in the x-direction measured in pixels, tₓ is the total number of pixels in the x-direction, and fₓ is the field of view of the camera in the x-direction measured in radians. It is noted that an equation similar to equation (18) above can be determined for y.

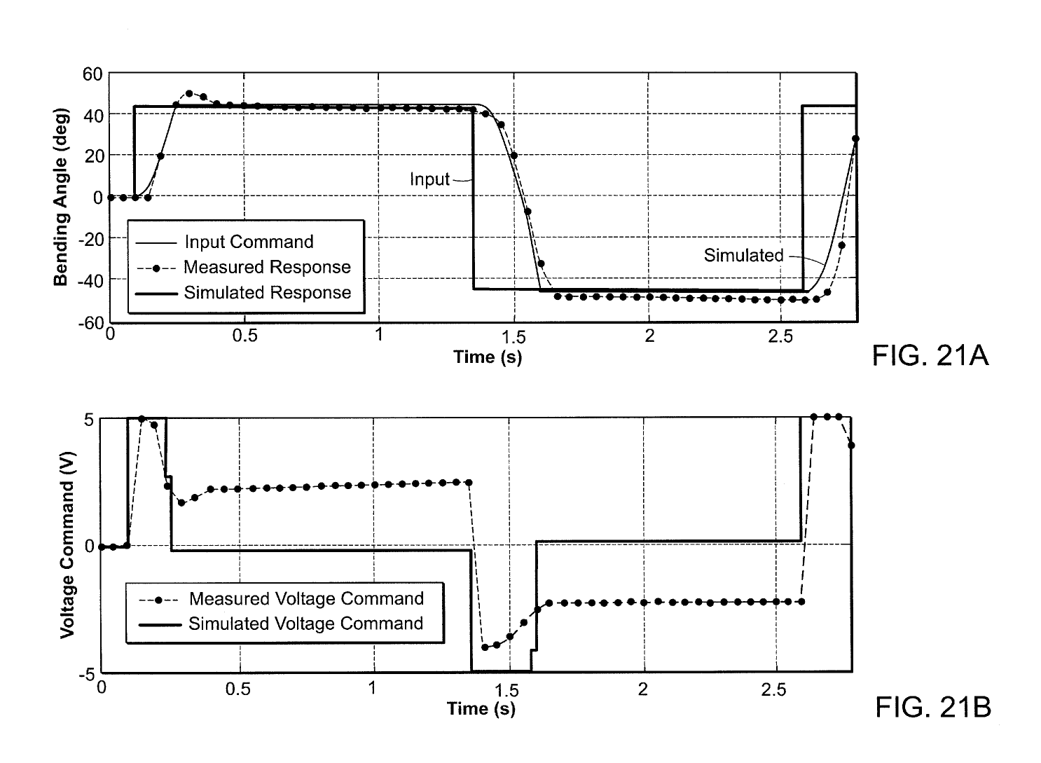

To validate the model described above, exemplary experiments were conducted on the gyroscope control loop, since this is the primary controller that is used by the tip angle control loop 1902 and the image stabilization control loop 1906. The measured and simulated results for a gain-scheduled proportional and derivative control scheme involving blind (i.e., unaided by the camera) turns of ±45 degrees are depicted in FIGS. 21A, 21B. In these exemplary experiments, gain scheduling is necessary because the frictional forces in the motor modules are not balanced for pulling in one direction versus the other. The gyroscopes Gyro 1, Gyro 2 periodically report the results to the user interface 1914. Despite the dead-band and windup delay nonlinearities, the maximum angular rotation rate for this design is about 400 degrees per second. Further, the windup delay at 45 degrees is about 50 ms, which is smaller than the delay caused by inertia and friction, as can be seen in FIG. 21A. FIG. 21B depicts exemplary voltage commands sent to the motors. The motor inputs, which are hard limited to between ±5 V, show matching patterns for the command structure. The overall trend, however, differs because the experimental system compensates for sensor drift, noise, and friction. The system achieves stability when the controller properly compensates for frictional losses.

While the present invention has been described herein in conjunction with preferred embodiment, a person of ordinary skill in the art, can effect changes, substitutions or equivalents to the systems and methods described herein, which are intended to fall within the appended claims and any equivalents thereof.

What is claimed is:

1. An endoscope comprising:
an endoscope body for insertion within a body lumen including an elongated endoscope body having a body module section, a motorized section and a flexible section, the motorized section being actuated to control deflection of the flexible section.

2. The endoscope of claim 1 further comprising a handle coupled to a proximal end of the endoscope body.

3. The endoscope of claim 1 further comprising a camera assembly mounted at a distal end of the endoscope.

4. The endoscope of claim 1 wherein the motorized section comprises a motor and a gear assembly.

5. The endoscope of claim 1 wherein the motorized section is coupled to the flexible section with a cable.

6. The endoscope of claim 5 wherein the motorized section comprises a second motor.

7. The endoscope of claim 2 wherein the handle is connected to a computer.

8. The endoscope of claim 2 wherein the handle comprises an actuator device that controls the motorized section.

9. The endoscope of claim 1 wherein the flexible section comprises a plurality of turning modules.

10. The endoscope of claim 9 further comprising a converter module that couples a motor module to a turning module.

11. The endoscope of claim 1 wherein the motorized section comprises a stepper motor connected to the flexible section with a cable and/or a flexible threaded rod.

12. The endoscope of claim 11 further comprising a second stepper motor connected to a second cable.

13. The endoscope of claim 1 wherein the flexible section is connected to the motorized section with a connector.

14. The endoscope of claim 1 wherein the body module section is connected to the motorized section with a connector.

15. The endoscope of claim 1 further comprising a distal housing connected to the flexible section with a connector.

16. The endoscope of claim 1 further comprising an actuator that adjusts rigidity of the body module section.

17. The endoscope of claim 16 wherein the body module section comprises a plurality of body modules connected to a tensioning wire.

18. The endoscope of claim 1 further comprising a motor actuator that controls deflection of the flexible section.

19. The endoscope of claim 5 wherein the motorized section comprises a motor that drives a shaft attached to a first end of the cable and a second end of the cable.

20. The endoscope of claim 1 further comprising a force sensor attached to the endoscope to measure deflection force of a distal end.

21. The endoscope of claim 15 further comprising a position sensor and/or an angle sensor and/or an angular velocity sensor that senses a position of the distal housing.

22. The endoscope of claim 1 further comprising a haptic feedback system.

23. The endoscope of claim 1 wherein a distal end of the motorized section is less than 20 mm from a proximal end of the flexible section.

24. The endoscope of claim 7 wherein the computer is programmed with a plurality of force levels.

25. The endoscope of claim 1 wherein the body module section is detachable from a handle and the motorized section.

26. The endoscope of claim 1 further comprising a gyroscope.

27. The endoscope of claim 26 wherein the gyroscope measures deflection angle of a distal tip of the endoscope.

28. The endoscope of claim 26 further comprising a second gyroscope that measures a second deflection angle of a distal tip of the endoscope.

29. The endoscope of claim 1 further comprising a control circuit mounted in the motorized section.

30. The endoscope of claim 29 wherein the control circuit comprises a motor control circuit.

31. The endoscope of claim 15 further comprising a distal control circuit in the distal housing.

32. The endoscope of claim 31 wherein the distal control circuit is connected to a handle control circuit and a motor control circuit in the motorized section is connected to the handle control circuit.

33. The endoscope of claim 15 further comprising a light source in the distal housing.

34. The endoscope of claim 4 wherein the gear assembly comprises a rotary to linear transmission.

35. The endoscope of claim 5 wherein the cable has length from a distal end of the motor module to a proximal end of the flexible module of less than 20 mm.

36. The endoscope of claim 35 wherein the length is less than 10 mm.

37. The endoscope of claim 1 further comprising a tubular outer sheath extending over the body module section, the motorized section, and the flexible section.

38. The endoscope of claim 1 further comprising a display connected to an imaging detector at the distal end of the endoscope.

39. The endoscope of claim 31 wherein the distal control circuit comprises a sensor circuit, a controller circuit board and a camera and light source circuit board.

40. The endoscope of claim 22 wherein the haptic feedback system comprises a user interface and haptic actuator in a handle.

41. (canceled)

42. The endoscope of claim 22 wherein the haptic control system comprises a force sensor connected to a resistance control system.

43. The endoscope of claim 1 further comprising a motor converter having a first disc connected to a second disc with a channel for fluid and a cable opening.

44. The endoscope of claim 1 further comprising a distal housing converter having a first disc connected to a second disc with channels for fluid and a cable opening.

45. The endoscope of claim 5 further comprising a safety module to detach the cable.

46. The endoscope of claim 4 wherein the gear assembly comprises a worm gear.

47. An endoscopic method comprising:
inserting an endoscope body into an object, the endoscope body including a body module section and a flexible section; and actuating a motorized section to control deflection of the flexible section.

48. The method of claim 47 further comprising performing a medical procedure with the endoscope.

49. The method of claim 47 further comprising illuminating a region of the object with a light source and detecting an image of the region with an imaging detector at a distal end of the endoscope.

50. The method of claim 48 further comprising detaching the body module section from a handle and attaching a second body module section to perform a second procedure.

51. The method of claim 47 further comprising measuring the deflection force.

52. The method of claim 47 further comprising measuring force of the flexible section and the speed.

53. The method of claim 47 further comprising programming a controller to limit maximum force exerted by the flexible section.

54-80. (canceled)
